(19) United States
(12) Patent Application Publication
(10) Pub. No.: US 2002/0058051 A1
(43) Pub. Date: May 16, 2002

(54) SKIN CARE COMPOSITIONS

(76) Inventors: ZAHID NAWAZ, LONDON (GB); EDWARD OWEN, SURREY (GB)

Correspondence Address: T DAVID REED, THE PROCTER & GAMBLE COMPANY, 5299 SPRING GROVE AVENUE, CINCINNATI, OH, 45217-1087, US

(21) Appl. No.: 09/142,120

(22) PCT Filed: Feb. 19, 1997
(86) PCT No.: PCT/US97/02555

(30) Foreign Application Priority Data
Mar. 5, 1996 (GB) 9604673.5

(51) Int. Cl. A61K 31/695; A61K 6/00; A61K 7/00; A61K 31/21; A61K 31/17

(52) U.S. Cl. 424/401; 514/63; 514/506; 514/588; 514/846; 514/938

(57) ABSTRACT

A skin care composition comprising. (a) a silicone-containing phase comprising cross-linked polyorganosiloxane polymer and silicone oil, wherein the composition comprises from about 0.1% to about 20% by weight of the combination of cross-linked polyorganosiloxane polymer and silicone oil, (b) from about 0.1% to about 20% by weight of an organic liquid crystal-forming amphiphilic surfactant, and (c) water; wherein the composition is in the form of an oil-in-water emulsion. The compositions of the invention provide improved skin feel, reduced greasiness/stickiness and faster absorption.

### TECHNICAL FIELD

The present invention relates to cosmetic compositions. In particular it relates to cosmetic compositions in the form of emulsions which provide improved moisturization, skin feel, skin care and appearance benefits and reduced greasiness, together with excellent rub-in and absorption characteristics. The compositions also display excellent stability characteristics at normal and elevated temperatures.

### BACKGROUND OF THE INVENTION

Skin is made up of several layers of cells which coat and protect the keratin and collagen fibrous proteins that form the skeleton of its structure. The outermost of these layers, referred to as the stratum corneum, is known to be composed of 25 nm protein bundles surrounded by 8 nm thick layers. Anionic surfactants and organic solvents typically penetrate the stratum corneum membrane and, by delipidization (i.e. removal of the lipids from the stratum corneum), destroy its integrity. This destruction of the skin surface topography leads to a rough feel and may eventually permit the surfactant or solvent to interact with the keratin, creating irritation.

It is now recognised that maintaining the proper water gradient across the stratum corneum is important to its functionality. Most of this water, which is sometimes considered to be the stratum corneum's plasticizer, comes from inside the body. If the humidity is too low, such as in a cold climate, insufficient water remains in the outer layers of the stratum corneum to properly plasticize the tissue, and the skin begins to scale and becomes itchy. Skin permeability is also decreased somewhat when there is inadequate water across the stratum corneum. On the other hand, too much water on the outside of the skin causes the stratum corneum to ultimately sorb three to five times its own weight of bound water. This swells and puckers the skin and results in approximately a two to three fold increase in the permeability of the skin to water and other polar molecules.

Thus, a need exists for compositions which will assist the stratum corneum in maintaining its barrier and water-retention functions at optimum performance in spite of deleterious interactions which the skin may encounter in washing, work, and recreation.

Conventional cosmetic cream and lotion compositions as described, for example, in Sagarin, Cosmetics Science and Technology, 2nd Edition, Vol.1, Wiley Interscience (1972) and Encyclopaedia of Chemical Technology, Third Edition, Volume 7 are known to provide varying degrees of emolliency, barrier and water-retention (moisturizing) benefits. However, they can also suffer serious negatives in terms of skin feel (i.e. they can feel very greasy on the skin), have poor rub-in and residue characteristics, and have slow absorption into the skin.

Thus, there remains a need for compositions which will assist the stratum corneum in maintaining its water gradient, but which do so with improved skin feel, rub-in and residue characteristics and absorption into the skin.

Compounds exhibiting liquid crystalline properties are known for use in skin care compositions. Liquid crystals constitute a particular phase which exists between the boundaries of the solid phase and the isotropic liquid phase (i.e. an intermediate between the three dimensionally ordered crystalline state and the disordered dissolved state). In the liquid crystal state, some of the molecular order characteristics of the solid phase are retained in the liquid state because of molecular association structure and long range intermolecular order. The ability of some compounds to form a liquid crystal phase had been observed nearly a century ago. Since that time many compounds exhibiting liquid crystalline properties have been synthesized and have been used to encapsulate and act as a delivery vehicle for drugs, flavours, nutrients and other compounds and for use in skin care compositions.

Silicone-based materials such as, for example, silicone fluids and silicone gums, are also well known for use in cosmetic compositions for providing benefits such as enhanced skin feel, application and spreadability characteristics. There is still a need however for improvements in skin feel, absorption characteristics and reduced tackiness/greasiness of the skin.

It has now been surprisingly found that by incorporating a particular type of material capable of forming liquid crystals into a cosmetic emulsion composition

It has now been surprisingly found that by incorporating a particular type of material capable of forming liquid crystals into a cosmetic emulsion composition comprising a silicone-containing phase which contains a crosslinked polyorganosiloxane polymer and silicone oil, a composition is provided which enhances moisturisation and skin feel and which in particular provides faster absorption and at the same time reduces stickiness and greasy feel on the skin.

### SUMMARY OF THE INVENTION

According to the present invention there is provided a skin care composition comprising:

(a) a silicone-containing phase comprising crosslinked polyorganosiloxane polymer and silicone oil, wherein the composition comprises from about 0.1% to about 20% by weight of the combination of said crosslinked polyorganosiloxane polymer and said silicone oil;

(b) from about 0.1% to about 20% by weight of a liquid crystal-forming amphiphilic surfactant; and

(c) water;

wherein the composition is in the form of an oil-in-water emulsion.

The compositions of the invention provide improved skin feel, reduced greasiness/stickiness and faster absorption.

### DETAILED DESCRIPTION OF THE INVENTION

The compositions of the present invention take the form of an oil-in-water emulsion containing one or more distinct emulsified oil phases together with an essential liquid crystal-forming surfactant component as well as various optional ingredients as indicated below. The compositions of the present invention essentially also contain a silicone-containing phase comprising crosslinked polyorganosiloxane polymer and silicone oil. All levels and ratios are by weight of total composition, unless otherwise expressly indicated. Chain length and degrees of ethoxylation are also specified on a weight average basis.

The term “skin conditioning agent”, as used herein means a material which provides a “skin conditioning benefit”. As used herein, the term “skin conditioning benefit” means any cosmetic conditioning benefit to the skin including, but not limited to, moisturization, humectancy (i.e. the ability to retain or hold water or moisture in the skin), emolliency, visual improvement of the skin surface, soothing of the skin, softening of the skin, improvement in skin feel, and the like.

The term “complete melting point”, as used herein means a melting point as measured by the well-known technique of Differential Scanning Calorimetry (DSC). The complete melting point is the temperature at the intersection of the baseline, i.e. the specific heat line, with the line tangent to the trailing edge of the endothermic peak. A scanning temperature of 5° C./minute is generally suitable in the present invention for measuring the complete melting points. However, it should be recognised that more frequent scanning rates may be deemed appropriate by the analytical chemist skilled in the art in specific circumsatnces. A DSC technique for measuring complete melting points is also described in U.S. Pat. No. 5,306,514, to Letton et al., issued, Apr. 26, 1994, incorporated herein by reference.

The term “nonocclusive” as used herein, means that the component as so described does not substantially obstruct or block up the passage of air and moisture through the skin surface.

A first essential component of the compositions herein is an oil or mixture of oils. In physical terms, the compositions generally take the form of an emulsion of one or more oil phases in an aqueous continuous phase, each oil phase comprising a single oily component or a mixture of oily components in miscible or homogeneous form, but said different oil phases containing different materials or combinations of materials from each other. The overall level of oil phase components in the compositions of the invention is preferably from about 0.1% to about 60%, more preferably from about 1% to about 30% and most preferably from about 1% to about 10% by weight.

The present compositions must comprise, as either all or a portion of the oil phase or oil phases referred to above a first silicone-containing phase comprising a crosslinked polyorganosiloxane polymer and a silicone oil, wherein the composition comprises 0.1% to about 20%, preferably from about 0.5% to about 10%, more preferably from about 0.5% to about 5%, by weight of composition, of the combination of crosslinked silicone and silicone oil.

The first silicone-containing phase comprises from about 10% to about 40%, more preferably from about 20% to about 30%, by weight of the first silicone-containing phase, of the crosslinked polyorganosiloxane polymer and from about 60% to about 90%, preferably from about 70% to about 80%, by weight of the first silicone-containing phase, of the silicone oil.

The crosslinked polyorganosiloxane polymer comprises polyorganosiloxane polymer crosslinked by a crosslinking agent. Crosslinking agents for use herein include any crosslinking agents useful for the preparation of crosslinked silicones. Suitable crosslinking agents herein include those represented by the following general formula:

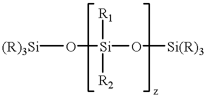

wherein R₁ is methyl, ethyl, propyl or phenyl, R₂ is H or —(CH₂)ₙCH═CH₂, where n is in the range of from about 1 to about 50, z is in the range of from about 1 to about 1000, preferably from about 1 to about 100 and R is an alkyl group having from 1 to 50 carbon atoms.

Preferably the crosslinking agent has the general formula

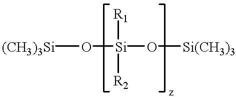

where R₁, R₂ and z are as defined above.

In especially preferred embodiments, the crosslinking agent has the following general formula:

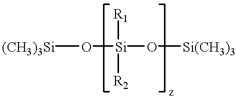

wherein z is in the range of from about 1 to about 1000, preferably from about 1 to about 100.

The crosslinked polysiloxane polymer preferably comprises from about 10% to about 50%, more preferably from about 20% to about 30%, by weight the crosslinked polysiloxane polymer, of crosslinking agent.

Any polyorganosiloxane polymers suitable for use in skin care compositions can be used herein. Suitable polyorganosiloxane polymers for use herein include those represented by the following general formula:

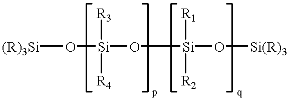

wherein R₁ is methyl, ethyl, propyl or phenyl, R₂ is H or —(CH₂)ₙCH═CH₂, where n is in the range of from about 1 to about 50, R₃ and R₄ are independently selected from methyl, ethyl, propyl and phenyl, R is an end-cap such as optionally hydroxy substituted alkyl group having from 1 to 50 carbon atoms, preferably an alkyl group having from 1 to 5 carbon atoms, more preferably an alkyl group having 1 or 2 carbon atoms, p is an integer in the range of from about 1 to about 2000, preferably from about 1 to about 500, q is an integer in the range of from about 1 to about 1000, preferably from about 1 to about 500.

In preferred embodiments the polyorganosiloxane is selected from polymers having the following general structure:

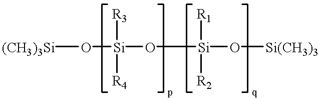

wherein R₁, R₂, R₃, R₄, p and q are as defined above.

As defined herein, p and q reflect the number of Si—O linkages in the polymer chain and R₁ and R₂ and R₃ and R₄ may vary going from one monomer unit to the next. For example, suitable polyorganosiloxane polymers for use herein include methyl vinyl dimethicone, methyl vinyl diphenyl dimethicone and methyl vinyl phenyl methyl diphenyl dimethicone.

In order to achieve crosslinking between the polyorganosiloxane polymer and the crosslinking agent, an (—Si—H) group must crosslink with a —Si—(CH₂)ₙCH═CH₂ group, so that for any specific crosslink, the group R₂ must be different in the polyorganosiloxane polymer and the crosslinking agent. For example, for any specific crosslink, when R₂ is —(CH₂)ₙCH═CH₂ in the polyorganosiloxane polymer, R₂ must be H in the crosslinking agent, and vice versa. However, there can be mixtures of R₂ for each of the polyorganosiloxane polymer and crosslinking agent.

In preferred embodiments, the polyorganosiloxane polymer is selected from an alkylarylpolysiloxane polymer having the general formula:

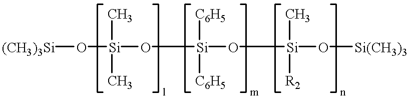

wherein R₂ is selected from —CH═CH₂ or H, preferably —CH═CH₂, and wherein 1 is an integer in the range of from about 1 to about 1000, preferably from about 1 to about 500, m is an integer in the range from 0 to about 1000, preferably from about 0 to about 500, and n is an integer in the range of from about 1 to about 1000, preferably from about 1 to about 100.

In particularly preferred embodiments the polyorganosiloxane polymer is selected from an alkylarylpolysiloxane polymer having the general formula:

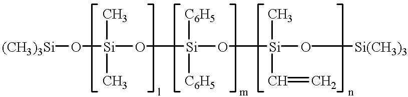

wherein 1, m and n are as defined above. In preferred embodiments m is in the range of from about 1 to about 1000, preferably from about 200 to about 800.

The first silicone-containing phase also comprises a silicone oil. Any straight chain, branched and cyclic silicones suitable for use in skin care compositions can be used herein. The silicone oils can be volatile or non-volatile. Suitable silicone oils for use herein include silicone oils having a weight average molecular weight of about 100,000 or less, preferably 50,000 or less. Preferably the silicone oil is selected from silicone oils having a weight average molecular weight in the range from about 100 to about 50,000, and preferably from about 200 to about 40,000. In preferred embodiments, the silicone oil is selected from dimethicone, decamethylcyclopentasiloxane, octamethylcyclotetrasiloxane and phenyl methicone, and mixtures thereof, most preferably phenyl methicone.

Suitable materials for use in the first silicone-containing phase are available under the tradename KSG supplied by Shinetsu Chemical Co., Ltd, for example KSG-15, KSG-16, KSG-17, KSG-18. These materials contain a combination of crosslinked polyorganosiloxane polymer and silicone oil. Particularly preferred for use herein especially in combination with the organic amphiphilic emulsifier material is KSG-18. The assigned INCI names for KSG-15, KSG-16, KSG-17 and KSG-18 are Cyclomethicone Dimethicone/Vinyl Dimethicone Crosspolymer, Dimethicone Dimethicone/Vinyl Dimethicone Crosspolymer, Cyclomethicone Dimethicone/Vinyl Dimethicone Crosspolymer and Phenyl Trimethicone Dimethicone/Phenyl Vinyl Dimethicone Crosspolymer, respectively.

Compositions herein preferably also comprise a second non-crosslinked silicone-containing phase. In preferred embodiments the second silicone-containing phase is present in a level of from about 0.1% to about 20%, especially from about 0.1% to about 10% by weight of composition.

Suitable silicone fluids for use in the second silicone-containing phase herein include water-insoluble silicones inclusive of non-volatile polyalkyl and polyaryl siloxane gums and fluids, volatile cyclic and linear polyalkylsiloxanes, polyalkoxylated silicones, amino and quaternary ammonium modified silicones, and mixtures thereof.

In preferred embodiments the second silicone-containing phase comprises a silicone gum or a mixture of silicones including the silicone gum. As used herein, the term “silicone gum” means high molecular weight silicone-based fluids having a mass-average molecular weight in excess of about 200,000 and preferably from about 200,000 to about 400,000. Generally, silicone oils have a molecular weight of less than about 200,000. Typically, silicone gums have a viscosity at 25° C. in excess of about 1,000,000 mm².s⁻¹. The silicone gums include dimethicones as described by Petrarch and others including U.S. Pat. No. 4,152,416, May 1, 1979 to Spitzer, et al, and Noll, Walter, Chemistry and Technology of Silicones New York: Academic Press 1968. Also describing silicone gums are General Electric Silicone Rubber Product Data Sheets SE 30, SE 33, SE 54 and SE 76.

Silicone gums for use herein include any silicone gum suitable for use in a skin care composition. Suitable silicone gums for use herein are silicone gums having a molecular weight of from about 200,000 to about 4,000,000 selected from dimethiconol, fluorosilicone and dimethicone and mixtures thereof.

Dimethiconol-based silicones suitable for use herein can have the chemical structure (II):

HO(CH₃)₂SiO[(CH₃)₂SiO]ₙ(CH₃)₂SiOH

where n is from about 2000 to about 40,000, preferably from about 3000 to about 30,000.

Exemplary fluorosilicones useful herein can have a molecular weight of from about 200,000 to about 300,000, preferably from about 240,000 to about 260,000 and most preferably about 250,000.

Specific examples of silicone gums include polydimethylsiloxane, (polydimethylsiloxane)(methylvinylsiloxane) copolymer, poly(dimethylsiloxane)(diphenyl)(methylvinylsiloxane) copolymer and mixtures thereof.

The silicone gum used herein can be incorporated into the composition as part of a mixture of silicones. When the silicone gum is incorporated as part of a mixture of silicones, the silicone gum preferably constitutes from about 5% to about 40%, especially from about 10% to 20% by weight of the silicone mixture. The silicone or silicone mixture preferably constitutes from about 0.1% to about 20%, more preferably from about 0.1% to about 15%, and especially from about 0.1% to about 10% by weight of composition.

Suitable silicone gum-based silicone mixtures for use in the second silicone-containing phase of the compositions herein include mixtures consisting essentially of:

(i) a silicone having a molecular weight of from about 200,000 to about 4,000,000 selected from dimethiconol, fluorosilicone and dimethicone and mixtures thereof; and

(ii) a silicone-based carrier having a viscosity from about 0.65 mm².s⁻¹ to about 100 mm².s⁻¹,

wherein the ratio of i) to ii) is from about 10:90 to about 20:80 and wherein said silicone gum-based component has a final viscosity of from about 500 mm².s⁻¹ to about 10,000 mm².s⁻¹.

of about 1,000,000 mm².s⁻¹.

The silicone-based carriers suitable for use herein include certain silicone fluids. The silicone fluid can be either a polyalkyl siloxane, a polyaryl siloxane, a polyalkylaryl siloxane or a polyether siloxane copolymer. Mixtures of these fluids can also be used and are preferred in certain executions.

The polyalkyl siloxane fluids that can be used include, for example, polydimethylsiloxanes with viscosities ranging from about 0.65 to 600,000 mm².s⁻¹, preferably from about 0.65 to about 10,000 mm².s⁻¹ at 25° C. These siloxanes are available, for example, from the General Electric Company as the Viscasil (RTM) series and from Dow Coming as the Dow Coming 200 series. The essentially non-volatile polyalkylarylsiloxane fluids that can be used include, for example, polymethylphenylsiloxanes, having viscosities of about 0.65 to 30,000 mm².s⁻¹ at 25° C. These siloxanes are available, for example, from the General Electric Company as SF 1075 methyl phenyl fluid or from Dow Coming as 556 Cosmetic Grade Fluid. Also suitable for use herein are certain volatile cyclic polydimethylsiloxanes having a ring structure incorporating from about 3 to about 7 (CH₃)₂SiO moieties.

The viscosity can be measured by means of a glass capillary viscometer as set forth in Dow Corning Corporate Test Method CTM0004, Jul. 29, 1970. Preferably the viscosity of the silicone blend constituting the second fluid phase ranges from about 500 mm2.s⁻¹ to about 100,000 mm².s⁻¹,.preferably from about 1000 mm².s⁻¹ to about 10,000 mm².s⁻¹.

An especially preferred silicone-gum based component for use in the compositions herein is a dimethiconol gum having a molecular weight of from about 200,000 to about 4,000,000 along with a silicone carrier with a viscosity of about 0.65 to 100 mm².s⁻¹. An example of this silicone component is Dow Coming Q2-1403 (85% 5 mm².s⁻¹ Dimethicone Fluid/15% Dimethiconol) and Dow Coming Q2-1401 available from Dow Coming.

Another class of silicone suitable for use in the second silicone-containing phase herein include polydiorganosiloxane-polyoxyalkylene copolymers containing at least one polydiorganosiloxane segment and at least one polyoxyalkylene segment, said polydiorganosiloxane segment consisting essentially of

R_{b}SiO_{(4-b)/2}

siloxane units wherein b has a value of from about 0 to about 3, inclusive, there being an average value of approximately 2 R radicals per silicon for all siloxane units in the copolymer, and R denotes a radical selected from methyl, ethyl, vinyl, phenyl and a divalent radical bonding said polyoxyalkylene segment to the polydiorganosiloxane segment, at least about 95% of all R radicals being methyl; and said polyoxyalkylene segment having an average molecular weight of at least about 1000 and consisting of from about 0 to about 50 mol percent polyoxypropylene units and from about 50 to about 100 mol percent polyoxyethylene units, at least one terminal portion of said polyoxyalkylene segment being bonded to said polydiorganosiloxane segment, any terminal portion of said polyoxyalkylene segment not bonded to said polydiorganosiloxane segment being satisfied by a terminating radical; the weight ratio of polydiorganosiloxane segments to polyoxyalkylene segments in said copolymer having a value of from about 2 to about 8. Such polymers are described in U.S. Pat. No. 4,268,499.

Preferred for use herein are polydiorganosiloxane-polyoxyalkylene copolymers having the general formula:

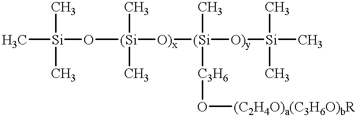

wherein x and y are selected such that the weight ratio of polydiorgano-siloxane segments to polyoxalkalkylene segments is from about 2 to about 8, the mol ratio of a:(a+b) is from about 0.5 to about 1, and R is a chain terminating group, especially selected from hydrogen; hydroxyl; alkyl, such as methyl, ethyl, propyl, butyl, benzyl; aryl, such as phenyl; alkoxy such as methoxy, ethoxy, propoxy, butoxy; benzyloxy; aryloxy, such as phenoxy; alkenyloxy, such as vinyloxy and allyloxy; acyloxy, such as acetoxy, acryloxy and propionoxy and amino, such as dimethylamino.

The number of and average molecular weights of the segments in the copolymer are such that the weight ratio of polydiorganosiloxane segments to polyoxyalkylene segments in the copolymer is preferably from about 2.5 to about 4.0.

Suitable copolymers are available commercially under the tradenames Belsil (RTM) from Wacker-Chemie GmbH, Geschäftsbereich S, Postfach D-8000 Munich 22 and Abil (RTM) from Th. Goldschmidt Ltd., Tego House, Victoria Road, Ruislip, Middlesex, HA40 YL, for example Belsil (RTM) 6031 and Abil (RTM) B88183. A particularly preferred copolymer for use herein includes Dow Corning DC3225C which has the CTFA designation Dimethicone/Dimethicone copolyol.

### Amphiphilic Surfactant

A further essential component of the compositions herein is an organic amphiphilic surfactant which is capable of forming smectic lyotropic crystals in product or when the product is being applied to the skin at ambient or elevated temperatures. Preferably the amphiphilic surfactant is capable of forming liquid crystals at a temperature in the range from about 200° C. to about 400° C. Preferably the amphiphilic surfactant is capable of forming smectic lyotropic liquid crystals. Once application of the product to the skin has been completed, liquid crystals may not be identifiable on the skin surface or stratum corneum. The amphiphilic surfactant is present at a level of from about 0.1% to about 20%, preferably from about 0.1% to about 10%, by weight.

The liquid-crystal forming amphiphilic surfactants suitable for use herein contain both hydrophilic and lipophilic groupings and exhibit a marked tendency to adsorb at a surface or interface, i.e. they are surface-active. Amphiphilic surface-active materials for use herein include nonionic (no charge), anionic (negative charge), cationic (positive charge) and amphoteric (both charges) based on whether or not they ionize in aqueous media.

In the literature, liquid crystals are also referred to as anisotropic fluids, a fourth state of matter, surfactant association structure or mesophases. Those terms are often used interchangeably. The term “lyotropic” means a liquid crystalline system containing a polar solvent, such as water. The liquid crystals used herein are preferably lamellar, hexagonal, rod or vesicle structures or mixtures thereof.

The liquid crystalline phase utilized in the compositions of the invention can be identified in various ways. A liquid crystal phase flows under shear and is characterised by a viscosity that is significantly different from the viscosity of its isotropic solution phase. Rigid gels do not flow under shear like liquid crystals. Also, when viewed with a polarized light microscope, liquid crystals show identifiable birefringence, as, for example, planar lamellar birefringence, whereas when isotropic solutions and rigid gels are viewed under polarized light, both show dark fields.

Other suitable means for identifying liquid crystals include X-ray diffraction, NMR spectroscopy and transmission electron microscopy.

In general terms, the organic amphiphilic surfactant preferred for use herein can be described as a liquid, semi-solid or waxy water-dispersible material having the formula X-Y where X represents a hydrophilic, especially nonionic moiety and Y represents a lipophilic moiety.

Organic amphiphilic surfactants suitable for use herein include those having a weight average HLB (Hydrophilic Lipophilic Balance) in the range from about 2 to about 12, preferably from about 4 to about 8.

Preferred organic amphiphilic surfactants employed herein have a long saturated or unsaturated branched or linear lipophilic chain having from about 12 to about 30 carbon atoms such as oleic, lanolic, tetradecylic, hexadecylic, isostearylic, lauric, coconut, stearic or alkyl phenyl chains. When the hydrophilic group of the amphiphilic material forming the liquid crystal phase is a nonionic group, a polyoxyethylene, a polyglycerol, a polyol ester, oxyalkylated or not, and, for example, a polyoxyalkylated sorbitol or sugar ester, can be employed. When the hydrophilic group of the amphiphilic material forming the liquid crystal phase is an ionic group, advantageously there can be used, as the hydrophilic group, a phosphatidylcholine residue as found in lecithin.

Hydrophilic moieties suitable for use herein are selected from:

(1) ethers of linear, or branched, polyglycerol having the following formula:

R—(Gly)ₙ—OH

wherein n is a whole number between 1 and 6, R is selected from aliphatic, linear or branched, saturated or unsaturated chains of 12 to 30 carbon atoms, the hydrocarbon radicals of lanolin alcohols and the 2-hydroxy alkyl residue of long chain, alpha-diols, and Gly represents a glycerol residue;

(2) polyethoxylated fatty alcohols, for example those of the formula R¹ (C₂ R₄O)ₓ OH wherein R¹ is C₁₂-C₃₀ linear or branched alkyl or alkenyl and x averages from about 0 to about 20, preferably from about 0.1 to about 6, more preferably from about 1 to about 4;

(3) polyol esters and polyalkoxylated polyol esters, and mixtures thereof, the polyols preferably being selected from sugars, C₂-C₆ alkylene glycols, glycerol, polyglycerols, sorbitol, sorbitan, polyethylene glycols and polypropylene glycols and wherein the polyalkoxylated polyol esters contain from about 2 to about 20 preferably from about 2 to about 4 moles of alkylene oxide (especially ethylene oxide) per mole of polyol ester;

(4) natural and synthetic phosphoglycerides, glycolipids and sphingolipids, for example cerebrosides, ceramides and lecithin.

Examples of amphiphilic surfactants suitable for use herein include C₈-C₃₀ alkyl and acyl-containing amphoteric, anionic, cationic and nonionic surfactants as set out below.

### Amphoteric

N-alkyl amino acids (e.g., sodium N-alkylaminoacetate);

N-lauroylglutamic acid cholesterol ester (e.g., Eldew CL-301 Ajinomoto)

### Anionic

Acylglutamates (e.g., disodium N-lauroylglutamate);

Sarcosinates (e.g., sodium lauryl sarcosinate. Grace, Seppic);

Taurates (e.g., sodium lauryl taurate. sodium methyl cocoyl taurate);

Carboxylic acids and salts (e.g., potassium oleate; potassium laurate;

potassium-10-undecenoate; potassium 11-(p-styryl)—undecanoate);

Ethoxylated carboxylic salts (e.g., sodium carboxy methyl alkyl ethoxylate);

Ether carboxylic acids;

Phosphoric acid esters and salts (e.g., lecithin; DEA-oleth-10 phosphate);

Acyl isethionates (e.g., sodium 2-lauroyloxyethane sulfonate);

Alkane sulfonates (e.g., branched sodium x-alkane sulfonate (x/1);

Sulfosuccinates e.g.,

Sodium dibutyl sulfosuccinate,

Sodium di-2-pentyl sulfosuccinate,

Sodium di-2-ethylbutyl sulfosuccinate,

Sodium di-hexyl-sulfosuccinate,

Sodium di-2 ethylhexyl sulfosuccinate (AOT),

Sodium di-2-ethyldodecyl sulfosuccinate,

Sodium di-2-ethyloctadecyl sulfosuccinate,

Dioctyl sodium sulfosuccinate,

Disodium laureth sulfosuccinate (MacKanate El, McIntyre Group Ltd.)

Sulfuric acid esters (e.g., sodium 2-ethylhept-6-enyl sulfate; sodium 11-heneicosyl sulfate; sodium 9-heptadecyl sulfate).

Alkyl sulfates (e.g., MEA alkyl sulfate such as MEA-lauryl sulfate)

### Cationic

Alkyl Imidazolines (e.g., alkyl hydroxyethyl imidazoline, stearyl hydroxyethyl imidazoline (supplier Akzo, Finetex and Hoechst));

Ethoxylated Amines (e.g., PEG-n alkylamine, PEG-n alkylamino propylamine, Poloxamine, PEG-cocopolyamine, PEG-15 tallow amine);

Alkylamines (e.g., dimethyl alkylamine; dihydroxyethyl alkylamine dioleate)

### Quaternaries

Alkylbenzyl dimethylammonium salts (e.g., stearalkonium chloride);

Alkyl betaines (e.g., dodecyl dimethyl ammonio acetate, oleyl betaine);

Heterocylic ammonium salts (e.g., alkylethyl morpholinium ethosulfate);

Tetraalkylammonium salts (e.g., dimethyl distearyl quaternary ammonium chloride (Witco));

Bis-isostearamidopropyl hydroxypropyl diammonium chloride (Schercoquat 21AP from Scher Chemicals);

1.8-Bis (decyldimethylammonio)-3, 6 dioxaoctane ditosylate

### Nonionic Surfactants

Ethoxylated glycerides;

Monoglycerides (e.g., monoolein; monolinolein; monolaurin; 1-dodecanoyl-glycerol monolaurin; 1, 13-docosenoyl-glycerol monoerucin diglyceride fatty acid (e.g., diglycerol monoisostearate Cosmol 41, fractionated. Nisshin Oil Mills Ltd.);

Polyglyceryl esters (e.g., triglycerol monooleate (Grindsted TS-T122), diglycerol monooleate (Grindsted TST-T101);

Polyhydric alcohol esters and ethers (e.g., sucrose cocoate, cetostearyl glucoside (Montanol, Seppic), β octyl glucofuranoside esters, alkyl glucoside such C₁₀-C₁₆ (Henkel));

Diesters of phosphoric acid (e.g., sodium dioleyl phosphate);

Alkylamido propyl betaine (e.g., cocoamido propyl betaine);

Amide: (e.g., N-(dodecanoylaminoethyl)-2-pyrrolidone);

Amide oxide; e.g., 1, 1 Dihydroperfluorooctyldimethylamine oxide, Dodecyldimethylamine oxide, 2-Hydroxydodecyldimethylamine oxide, 2-Hydroxydodecyl-bis (2-hydroxyethyl) amine oxide, 2-Hydroxy-4-oxahexadecyldimethylamine oxide,

Ethoxylated amides (e.g., PEG-n acylamide);

Ammonio phosphates (e.g., didecanoyl lecithin);

Amine (e.g., octylamine); Ammonio amides e.g., N-trimethylammoniodecanamidate, N-trimethylammoniododecanamidate,

Ammonio carboxylates e.g., dodecyldimethylammonioacetate, 6-didodecylmethylammoniohexanoate,

Phosphonic and phosphoric esters and amides e.g., methyl-N-methyl-dodecylphosphonamidate, dimethyl dodecylphosphonate, dodecyl methyl methylphosphonate, N,N-dimethyl dodecylphosphonic diamide

### Ethoxylated Alcohols

Polyoxyethylene (C₈) e.g., pentaoxyethylene glycol p-n-octylphenyl ether hexaoxyethylene glycol p-n-octylphenyl ether nonaoxyethylene glycol p-n-octylphenyl ether

Polyoxyethylene (C₁₀) e.g., pentaoxyethylene glycol p-n-decylphenyl ether, decyl glyceryl ether, 4-oxatetradecan-1, 2-diol, nonaoxyethylene glycol p-n-decylphenyl ether

Polyoxyethylene (C₁₁) e.g., Tetraoxyethylene glycol undecyl ether

Polyoxyethylene (C₁₂) e.g., 3, 6, 9, 13-tetraoxapentacosan 1, 11-diol, 3, 6, 10-trioradocosan-1, 8-diol, 3, 6, 9, 12, 16-pentaoxaoctacosan 1, 14-diol, 3, 6, 9, 12, 15-pentaoxanonacosan-1, 17-diol, 3, 7-dioxanonadecan-1, 5-diol, 3, 6, 12, 15, 19-hexaoxahentriacontan-1, 16-diol, pentaoxyethylene glycol dodecyl ether, monaoxyethylene glycol p-n-dodecylphenyl ether,

Polyoxyethylene(C₁₄) e.g., 3, 6, 9, 12, 16-pentaoxaoctacosan-1, 14-diol, 3, 6, 9, 12, 15, 19-heraoxatriacontan-1, 17-diol,

Sulfone diimines e.g., decyl methyl sulfone diimine

Sulfoxides e.g., 3-decyloxy-2-hydroxypropyl methyl sulfoxide 4-decyloxy-3-hydroxybutyl methyl sulfoxide

Sulfoximines e.g., N-methyl dodecyl methyl sulfoximine

Preferred organic amphiphilic surfactants for use herein are nonionic amphiphilic surfactants having a hydrophilic moiety selected from polyol esters and polyalkoxylated polyol esters, and mixtures thereof, the polyols preferably being selected from sugars, C₂-C₆ alkylene glycols, glycerol, polyglycerols, sorbitol, sorbitan, polyethylene glycols and polypropylene glycols and wherein the polyalkoxylated polyol esters contain from about 2 to about 20 preferably from about 2 to about 4 moles of alkylene oxide (especially ethylene oxide) per mole of polyol ester, and a lipophilic moiety selected from long saturated or unsaturated branched chain or linear lipophilic chains having from about 12 to about 30 carbon atoms such as oleic, lanolic, tetradecylic, hexadecylic, isostearylic, lauric, coconut, stearic or alkyl phenyl chains.

Highly preferred organic amphiphilic surfactants for use herein are selected from polyhydric alcohol esters and ethers. Especially preferred amphiphilic surfactants for use herein are sugar esters and polyalkoxylated sugar esters.

The sugar esters for use in this invention can be classified as hydrocarbyl and alkyl polyoxyalkylene esters of cyclic polyhydroxy saccharides wherein one or more of the hydroxyl groups on the saccharide moiety is substituted with an acyl or polyoxyalkylene group. Hydrocarbyl sugar esters can be prepared in well-known fashion by heating an acid or acid halide with sugar, i.e., by a simple esterification reaction.

The sugars employed in the preparation of the sugar esters include monosaccharides, di-saccharides and oligo-saccharides well known in the art, for example, the dextrorotatory and levorotatory forms of glucose, fructose, mannose, galactose, arabinose and xylose. Typical di-saccharides include maltose, cellibiose, lactose, and trehalose. Typical tri-saccharides include raffinose and gentianose. The di-saccharides are preferred for use herein, especially sucrose.

Sucrose can be esterified at one or more of its eight hydroxyl groups to provide the sucrose esters useful herein. When sucrose is combined with an esterification agent in a 1:1 mole ratio, sucrose monoesters are formed; when the ratio of esterification agent to sucrose is 2: 1, or greater, the di-, tri-, etc., esters are formed, up to a maximum of the octa-ester.

Preferred sugar esters herein are those prepared by the esterification of sugars at a mole ratio of esterification agent: sugar of 1:1 and 3:1 i.e., the mono-acyl and di- or higher acyl sugar esters. Especially preferred are the mono-, di- and tri-acyl sugar esters and mixtures thereof wherein the acyl substituents contain from about 8 to about 24, preferably from about 8 to about 20 carbon atoms and 0,1 or 2 unsaturated moieties. Of the mono-acyl and di-acyl sugar esters, the respective esters of di-saccharide sugars, especially sucrose, wherein the acyl groups contain from about 8 to about 20 carbon atoms are especially preferred. Preferred sugar esters herein are sucrose cocoate, sucrose monooctanoate, sucrose monodecanoate, sucrose monolaurate, sucrose monomyristate, sucrose monopalmitate, sucrose monostearate, sucrose monooleate, sucrose monolinoleate, sucrose dioleate, sucrose dipalmitate, sucrose distearate, sucrose dilaurate and sucrose dilinoleate, and mixtures thereof. Sucrose cocoate has been found to be particularly efficacious in the compositions herein. In mixtures of mono-acyl with di-, tri- and higher acyl sugar esters, the mono- and di-acyl esters preferably comprise at least about 40%, more preferably from about 50% to about 95% by weight of the total sugar ester mixture.

Other sugar esters suitable for use in the compositions of this invention are the alkyl polyoxyalkylene sugar esters wherein one hydroxyl group is substituted with a C₈-C₁₈ alkyl group and wherein one or more of the hydroxyl groups on the sugar molecule are replaced by an ester or ether substituent containing the moiety [(CH₂)ₓ—O]_{y} wherein x is an integer from 2 to about 4, preferably 2, and wherein y is an integer from about 1 to about 50, preferably 8 to 30 polyoxyalkylene substituents. Especially preferred herein are sugar esters wherein the polyoxyalkylene substituent is a polyoxyethylene substituent containing from about 8 to about 30 polyoxyethylene groups. Such materials wherein sorbitan is the sugar moiety are commercially available under the tradename “Tweens”. Such mixed esters can be prepared by first acylating a sugar at a 1:1 mole ratio with a hydrocarbyl acid halide followed by reaction with the corresponding polyoxyalkylene acid halide or alkylene oxide to provide the desired material. The simple polyoxyalkylene ester of di-saccharides, especially sucrose, wherein the polyoxyalkylene groups contain up to about 20 alkylene oxide moieties are another useful class of sugar esters herein. A preferred sugar ester of this class is sorbitol trioleate ethoxylated with 20 moles of ethylene oxide. Mixtures of sugar esters with other polyol esters, eg. glycerol esters, are also suitable for use herein, for example, Palm Oil Sucroglyceride (Rhone-Poulenc).

As used herein, the term “lecithin” refers to a material which is a phosphatide. Naturally occurring or synthetic phosphatides can be used. Phosphatidylcholine or lecithin is a glycerine esterified with a choline ester of phosphoric acid and two fatty acids, usually a long chain saturated or unsaturated fatty acid having 16-20 carbons and up to 4 double bonds. Other phosphatides capable of forming lamellar or hexagonal liquid crystals can be used in place of the lecithin or in combination with it. These phosphatides are glycerol esters with two fatty acids as in the lecithin, but the choline is replaced by ethanolamine (a cephalin), or serine (-aminopropanoic acid; phosphatidyl serine) or an inositol (phosphatidyl inositol). While the invention herein is exemplified with lecithin, it is understood that these other phosphatides can be used herein.

A variety of lecithins can be used. American Lecithin Company supplies a Nattermann Phospholipid, Phospholipan 80 and Phosal 75. Other lecithins which can be used alone or in combination with these are: Actifla Series, Centrocap series, Central Ca, Centrol series, Centrolene, Centrolex, Centromix, Centrophase and Centrolphil Series from Central Soya; Alcolec and Alcolec 439-C from American Lecithin; Canaspersa from Canada Packers, Lexin K and Natipide from American Lecithin; and L-Clearate, Clearate LV and Clearate WD from the W.A. Cleary Co. Lecithins are supplied dissolved in ethanol, fatty acids, triglycerides and other solvents. They are usually mixtures of lecithins and range from 15% to 50% of the solution as supplied.

Both natural and synthetic lecithins can be used. Natural lecithins are derived from oilseeds such as sunflower seeds, soybeans, safflower seeds and cottonseed. The lecithins are separated from the oil during the refining process.

The organic amphiphilic surfactant has been found to be especially valuable herein for improving the stability and skin feel of the compositions of the invention.

The amphiphilic surfactant is preferably incorporated into the composition in an amount of from about 0.1% to about 20% , preferably from about 0.1% to about 10%, and more preferably from about 0.1% to about 8% by weight of composition.

Highly preferred herein is a fatty acid ester blend based on a mixture of sorbitan or sorbitol fatty acid ester and sucrose fatty acid ester, the fatty acid in each instance being preferably C₈-C₂₄, more preferably C₁₀-C₂₀. The preferred fatty acid ester from the viewpoint of moisturisation is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the trade name Arlatone 2121.

### Optional Ingredients

In preferred embodiments, a third oil phase is present in an amount of from about 0.1% to about 15%, more preferably from about 1% to about 10% by weight of composition. The third oil phase can be either a separate phase or can form one phase together with either or both of the first and second silicon phases. Preferably, the third oil phase is a separate phase.

The third oil phase preferably comprises a non-silicone organic oil, such as a natural or synthetic oil selected from mineral, vegetable, and animal oils, fats and waxes, fatty acid esters, fatty alcohols, fatty acids and mixtures thereof, which ingredients are useful for achieving emollient cosmetic properties. The first oil phase component is preferably essentially silicone-free, i.e., it contains no more than about 10%, preferably no more than about 5% by weight of silicone-based materials. It will be understood that the oil phase may contain, for example, up to about 25%, preferably up to only about 10% of oil phase soluble emulsifier ingredients. Such ingredients are not to be considered as oil phase components from the viewpoint of determining the oil phase level and required HLB. In preferred embodiments, the overall required HLB of the oil phase is from about 8 to about 12, especially from about 9 to about 11, required HLB being determined by summing the individual required HLB values for each component of the oil phase multiplied by its W/W percentage in the oil phase (see ICI Literature on HLB system; ICI reference paper ref 51/0010/303/15 m., first printed in 1976, revised in 1984 and May 1992).

Suitable first oil phase components for use herein include, for example, optionally hydroxy-substituted C₈-C₅₀ unsaturated fatty acids and esters thereof, C₁-C₂₄ esters of C₈-C₃₀ saturated fatty acids such as isopropyl myristate, isopropyl palmitate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum and squalane, fatty sorbitan esters (see U.S. Pat. No. 3988255, Seiden, issued Oct. 26 1976), lanolin and lanolin derivatives, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, shea butter, shorea butter, and sunflower seed oil and C₁-C₂₄ esters of dimer and trimer acids such as diisopropyl dimerate, diisostearylmalate, diisostearyldimerate and triisostearyltrimerate. Of the above, highly preferred are the mineral oils, petrolatums, unsaturated fatty acids and esters thereof and mixtures thereof.

Preferred embodiments herein comprise from about 0.1% to about 10% by weight of an unsaturated fatty acid or ester. Preferred unsaturated fatty acids and esters for use herein are optionally hydroxy substituted C₈-C₅₀ unsaturated fatty acids and esters, especially esters of ricinoleic acid. The unsaturated fatty acid or ester component is valuable herein in combination with the liquid crystal-forming emulsifier for improving the skin feel and rub-in characteristics of the compositon. Highly preferred in this respect is cetyl ricinoleate.

A preferred component of the compositions herein, in addition to the organic amphiphilic surfactant is a polyol ester skin conditioning agent.

The compositions of the present invention preferably comprise from about 0.01% to about 20%, more preferably from about 0.1% to about 15%, and especially from about 1% to about 10% by weight of the polyol ester. The level of polyol ester by weight of the oil in the composition is preferably from about 1% to about 30%, more preferably from about 5% to about 20%.

The polyol ester preferred for use herein is a nonocclusive liquid or liquifiable polyol carboxylic acid ester. These polyol esters are derived from a polyol radical or moiety and one or more carboxylic acid radicals or moieties. In other words, these esters contain a moiety derived from a polyol and one or more moieties derived from a carboxylic acid. These carboxylic acid esters can also be derived from a carboxylic acid. These carboxylic acid esters can also be described as liquid polyol fatty acid esters, because the terms carboxylic acid and fatty acid are often used interchangeably by those skilled in the art.

The preferred liquid polyol polyesters employed in this invention comprise certain polyols, especially sugars or sugar alcohols, esterified with at least four fatty acid groups. Accordingly, the polyol starting material must have at least four esterifiable hydroxyl groups. Examples of preferred polyols are sugars, including monosaccharaides and disaccharides, and sugar alcohols. Examples of monosaccharides containing four hydroxyl groups are xylose and arabinose and the sugar alcohol derived from xylose, which has five hydroxyl groups, i.e., xylitol. The monosaccharide, erythrose, is not suitable in the practice of this invention since it only contains three hydroxyl groups, but the sugar alcohol derived from erythrose, i.e., erythritol, contains four hydroxyl groups and accordingly can be used. Suitable five hydroxyl group-containing monosaccharides are galactose, fructose, and sorbose. Sugar alcohols containing six —OH groups derived from the hydrolysis products of sucrose, as well as glucose and sorbose, e.g., sorbitol, are also suitable. Examples of disaccharide polyols which can be used include maltose, lactose, and sucrose, all of which contain eight hydroxyl groups.

Preferred polyols for preparing the polyesters for use in the present invention are selected from the group consisting of erythritol, xylitol, sorbitol, glucose, and sucrose. Sucrose is especially preferred.

The polyol starting material having at least four hydroxyl groups is esterified on at least four of the —OH groups with a fatty acid containing from about 8 to about 22 carbon atoms. Examples of such fatty acids include caprylic, capric, lauric, myristic, myristoleic, palmitic, palmitoleic, stearic, oleic, ricinoleic, linoleic, linolenic, eleostearic, arachidic, arachidonic, behenic, and erucic acid. The fatty acids can be derived from naturally occurring or synthetic fatty acids; they can be saturated or unsaturated, including positional and geometrical isomers. However, in order to provide liquid polyesters preferred for use herein, at least about 50% by weight of the fatty acid incorporated into the polyester molecule should be unsaturated. Oleic and linoleic acids, and mixtures thereof, are especially preferred.

The polyol fatty acid polyesters useful in this invention should contain at least four fatty acid ester groups. It is not necessary that all of the hydroxyl groups of the polyol be esterified with fatty acid, but it is preferable that the polyester contain no more than two unesterified hydroxyl groups. Most preferably, substantially all of the hydroxyl groups of the polyol are esterified with fatty acid, i.e., the polyol moiety is substantially completely esterified. The fatty acids esterified to the polyol molecule can be the same or mixed, but as noted above, a substantial amount of the unsaturated acid ester groups must be present to provide liquidity.

To illustrate the above points, a sucrose fatty triester would not be suitable for use herein because it does not contain the required four fatty acid ester groups. A sucrose tetra-fatty acid ester would be suitable, but is not preferred because it has more than two unesterified hydroxyl groups. A sucrose hexa-fatty acid ester would be preferred because it has no more than two unesterified hydroxyl groups. Highly preferred compounds in which all the hydroxyl groups are esterified with fatty acids include the liquid sucrose octa-substituted fatty acid esters.

The following are non-limiting examples of specific polyol fatty acid polyesters containing at least four fatty acid ester groups suitable for use in the present invention: glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof.

As noted above, highly preferred polyol fatty acid esters are those wherein the fatty acids contain from about 14 to about 18 carbon atoms.

The preferred liquid polyol polyesters preferred for use herein have complete melting points below about 30° C., preferably below about 27.5° C., more preferably below about 25° C. Complete melting points reported herein are measured by Differential Scanning Calorimetry (DSC).

The polyol fatty acid polyesters suitable for use herein can be prepared by a variety of methods well known to those skilled in the art. These methods include: transesterification of the polyol with methyl, ethyl or glycerol fatty acid esters using a variety of catalysts; acylation of the polyol with a fatty acid chloride; acylation of the polyol with a fatty acid anhydride; and acylation of the polyol with a fatty acid, per se. See U.S. Pat. No. 2,831,854; U.S. Pat. No. 4,005,196, to Jandacek, issued Jan. 25, 1977; U.S. Pat. No. 4,005,196, to Jandacek, issued Jan. 25, 1977.

A highly preferred ingredient of the compositions herein is urea which is preferably present in a level of from about 0.1% to about 20%, more preferably from about 0.5% to about 10% and especially from about 1% to about 5% by weight of composition.

In preferred embodiments, the oil phase and organic amphiphilic material are premixed in water at a temperature above the Kraft Point of the organic amphiphilic material (but preferably below about 60° C.) to form a liquid crystal/oil in water dispersion prior to addition of the urea. The urea is found to be especially effective herein in combination with the amphiphilic emulsifier material and the polyol fatty acid polyester for providing outstanding skin moisturisation and softening in the context of an oil-in-water skin care emulsion composition. Moreover, it is surprisingly found that the urea is rendered more stable to hydrolytic degradation, thereby allowing an increase in compositional pH.

A wide variety of optional ingredients such as non-occlusive moisturizers, humectants, gelling agents, neutralizing agents, perfumes, coloring agents and surfactants, can be added to the skin compositions herein.

The compositions herein can comprise a humectant. Suitable humectants for use herein include sorbitol, propylene glycol, butylene glycol, hexylene glycol, ethoxylated glucose derivatives, hexanetriol, glycerine, water-soluble polyglycerylmethacrylate lubricants, glycine, hyaluronic acid, arginine, Ajidew (NaPCA) and panthenols. A preferred humectant herein is glycerine (sometimes known as glycerol or glycerin). Chemically, glycerine is 1,2,3-propanetriol and is a product of commerce. One large source of the material is in the manufacture of soap. Glycerine is especially preferred in the compositions of the invention from the viewpoint of boosting moisturisation. Also preferred for use herein is butylene glycol. Particularly preferred from the viewpoint of boosting moisturisation is a combination of glycerine and urea.

In the present compositions, the humectant is preferably present at a level of from about 0.1% to about 20%, more preferably from about 1% to about 15%, and especially from about 5% to about 15% by weight of composition.

Suitable polyglycerylmethacrylate lubricants for use in the compositions of this invention are available under the trademark Lubrajel (RTM) from Guardian Chemical Corporation, 230 Marcus Blvd., Hauppage, N.Y. 11787. In general, Lubrajels can be described as hydrates or clathrates which are formed by the reaction of sodium glycerate with a methacrylic acid polymer. Thereafter, the hydrate or clathrate is stabilized with a small amount of propylene glycol, followed by controlled hydration of the resulting product. Lubrajels are marketed in a number of grades of varying glycerate: polymer ratio and viscosity. Suitable Lubrajels include Lubrajel TW, Lubrajel CG and Lubrajel MS, Lubrajel WA, Lubrajel DV and so-called Lubrajel Oil.

At least part (up to about 5% by weight of composition) of the humectant can be incorporated in the form of an admixture with a particulate lipophilic or hydrophobic carrier material. The carrier material and humectant can be added either to the aqueous or disperse phase.

This copolymer is particularly valuable for reducing shine and controlling oil while helping to provide effective moisturization benefits. The cross-linked hydrophobic polymer is preferably in the form of a copolymer lattice with at least one active ingredient dispersed uniformly throughout and entrapped within the copolymer lattice. Alternatively, the hydrophobic polymer can take the form of a porous particle having a surface area (N₂,BET) in the range from about 50 to 500, preferably 100 to 300m².g⁻¹ and having the active ingredient absorbed therein.

The cross-linked hydrophobic polymer is preferably present in an amount of from about 0.1% to about 10% by weight and is preferably incorporated in the external aqueous phase. The active ingredient can be one or more or a mixture of skin compatible oils, skin compatible humectants, emollients, moisturizing agents and sunscreens. In one embodiment, the polymer material is in the form of a powder, the powder being a combined system of particles. The system of powder particles forms a lattice which includes unit particles of less than about one micron in average diameter, agglomerates of fused unit particles of sized in the range of about 20 to 100 microns in average diameter and aggregates of clusters of fused agglomerates of sizes in the range of about 200 to 1,200 microns in average diameter.

The powder material of this embodiment can be broadly described as a cross-linked “post absorbed” hydrophobic polymer lattice. The powder preferably has entrapped and dispersed therein, an active which may be in the form of a solid, liquid or gas. The lattice is in particulate form and constitutes free flowing discrete solid particles when loaded with the active material. The lattice may contain a predetermined quantity of the active material. A suitable polymer has the structural formula:

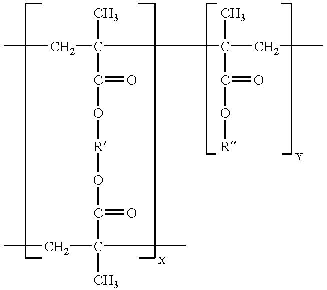

where the ratio of x to y is 80:20, R′ is —CH₂CH₂— and R″ is —(CH₂)₁₁CH₃.

The hydrophobic polymer is a highly crosslinked polymer, more particularly a highly cross-linked polymethacrylate copolymer. The material is manufactured by the Dow Coming Corporation, Midland. Mich., USA, and sold under the trademark POLYTRAP (RTM). It is an ultralight free-flowing white powder and the particles are capable of absorbing high levels of lipophilic liquids and some hydrophilic liquids while at the same time maintaining a free-flowing powder character. The powder structure consists of a lattice of unit particles less than one micron that are fused into agglomerates of 20 to 100 microns and the agglomerates are loosely clustered into macro-particles or aggregates of about 200 to about 1200 micron size. The polymer powder is capable of containing as much as four times its weight of fluids, emulsions, dispersion or melted solids.

Adsorption of actives onto the polymer powder can be accomplished using a stainless steel mixing bowl and a spoon, wherein the active is added to the powder and the spoon is used to gently fold the active into the polymer powder. Low viscosity fluids may be adsorbed by addition of the fluids to a sealable vessel containing the polymer and then tumbling the materials until a consistency is achieved. More elaborate blending equipment such as ribbon or twin cone blenders can also be employed. The preferred active ingredient for use herein is glycerine. Preferably, the weight ratio of humectant : carrier is from about 1:4 to about 3:1.

Also suitable as a highly cross-linked polymethacrylate copolymer is Microsponges 5647. This takes the form of generally spherical particles of cross-linked hydrophobic polymer having a pore size of from about 0.01 to about 0.05 μm and a surface area of 200-300 m²/g. Again, it is preferably loaded with humectant in the levels described above.

The compositions of the invention can also contain a hydrophilic gelling agent at a level preferably from about 0.01% to about 10%, more preferably from about 0.02% to about 2%, and especially from about 0.02% to about 0.5%. The gelling agent preferably has a viscosity (1% aqueous solution, 20° C., Brookfield RVT) of at least about 4000 mPa.s, more preferably at least about 10,000 mPa.s and especially at least 50,000 mPa.s.

Suitable hydrophilic gelling agents can generally be described as water-soluble or colloidally water-soluble polymers, and include cellulose ethers (e.g. hydroxyethyl cellulose, methyl cellulose, hydroxypropylmethyl cellulose), polyvinylpyrrolidone, polyvinylalcohol, guar gum, hydroxypropyl guar gum and xanthan gum.

Preferred hydrophilic gelling agents herein, however, are acrylic acid/ethyl acrylate copolymers and the carboxyvinyl polymers sold by the B.F. Goodrich Company under the trade mark of Carbopol resins. These resins consist essentially of a colloidally water-soluble polyalkenyl polyether crosslinked polymer of acrylic acid crosslinked with from 0.75% to 2.00% of a crosslinking agent such as for example polyallyl sucrose or polyallyl pentaerythritol. Examples include Carbopol 934, Carbopol 940, Carbopol 950, Carbopol 954, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule. A most preferred polymer is Carbopol 954. Also suitable for use herein are hydrophobically-modified cross-linked polymers of acrylic acid having amphipathic properties available under the Trade Name Carbopol 1382, Carbopol 1342 and Pemulen TR-1 (CTFA Designation: Acrylates/10-30 Alkyl Acrylate Crosspolymer). A combination of the polyalkenyl polyether cross-linked acrylic acid polymer and the hydrophobically modified cross-linked acrylic acid polymer is also suitable and is preferred for use herein. The gelling agents herein are particularly valuable for providing excellent stability characteristics over both normal and elevated temperatures.

Neutralizing agents suitable for use in neutralizing acidic group containing hydrophilic gelling agents herein include sodium hydroxide, potassium hydroxide, ammonium hydroxide, monoethanolamine, diethanolamine and triethanolamine.

The compositions of the invention are in emulsion form and are preferably formulated so as to have a product viscosity of at least about 4,000 mPa.s and preferably in the range from about 4,000 to about 300,000 mPa.s, more preferably from about 8,000 to about 200,000 mPa.s and especially from about 10,000 to about 100,000 mPa.s and even more especially from about 10,000 to about 50,000 mPa.s (25° C., neat, Brookfield RVT Spindle No. 5).

The compositions of the invention can also contain from about 0.1% to about 10%, preferably from about 1% to about 5% of a panthenol moisturizer. The panthenol moisturizer can be selected from D-panthenol ([R]-2,4-dihydroxy-N-[3-hydroxypropyl)]-3,3-dimethylbutamide), DL-panthenol, calcium pantothenate, royal jelly, panthetine, pantotheine, panthenyl ethyl ether, pangamic acid, pyridoxin, pantoyl lactose and Vitamin B complex. Highly preferred from the viewpoint of skin care and tack reduction is D-panthenol.

The compositions of the present invention can additionally comprise from about 0.001% to about 0.5%, preferably from about 0.002% to about 0.05%, more preferably from about 0.005% to about 0.02% by weight of carboxymethylchitin. Chitin is a polysaccharide which is present in the integument of lobsters and crabs and is a mucopolysaccharide having beta (1-4) linkages of N-acetyl-D-glucosamine. Carboxymethylchitin is prepared by treating the purified chitin material with alkali followed by monochloracetic acid. It is sold commercially in the form of a dilute (approximately 0.1% to 0.5% by weight) aqueous solution under the name Chitin Liquid available from A & E Connock Ltd., Fordingbridge, Hampshire, England.

Other optional materials include keratolytic agents such as salicylic acid; proteins and polypeptides and derivatives thereof; water-soluble or solubilizable preservatives such as Germall 115, methyl, ethyl, propyl and butyl esters of hydroxybenzoic acid, benzyl alcohol, EDTA, Euxyl (RTM) K400, Bromopol (2-bromo-2-nitropropane- 1,3-diol) and phenoxypropanol; anti-bacterials such as Irgasan (RTM) and phenoxyethanol (preferably at levels of from 0.1% to about 5%); soluble or colloidally-soluble moisturising agents such as hylaronic acid and starch-grafted sodium polyacrylates such as Sanwet (RTM) IM-1000, IM-1500 and IM-2500 available from Celanese Superabsorbent Materials, Portsmith, Va., USA and described in U.S. Pat. No. 4,076,663; vitamins such as vitamin A, vitamin C, vitamin E and vitamin K; alpha and beta hydroxyacids; aloe vera; sphingosines and phytosphingosines, cholesterol; skin whitening agents; N-acetyl cysteine; coloring agents; perfumes and perfume solubilizers and additional surfactants/emulsifiers such as fatty alcohol ethoxylates, ethoxylated polyol fatty acid esters, wherein the polyol can be selected from glycerine, propyleneglycol, ethyleneglycol, sorbitol, sorbitan, polypropyleneglycol, glucose and sucrose. Examples include glyceryl monohydroxy stearate and stearyl alcohol ethoxylated with an average of from 10 to 200 moles of ethyleneoxide per mole of alcohol and PEG-6 caprylic/capric glycerides.

Also useful herein are sunscreening agents. A wide variety of sunscreening agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology. Preferred among those sunscreens which are useful in the compositions of the instant invention are those selected from 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, Parsol MCX, Eusolex 6300, Octocrylene, Parsol 1789 and mixtures thereof.

Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991. The sunscreening agents disclosed therein have, in a single molecule, two distinct chromophore moieties which exhibit different ultra-violet radiation absorption spectra. One of the chromophore moieties absorbs pre-dominantly in the UVB radiation range and the other absorbs strongly in the UVA radiation range. These sunscreening agents provide higher efficacy, broader UV absorption, lower skin penetration and longer lasting efficacy relative to conventional sunscreens. Especially preferred examples of these sunscreens include those selected from 4-N,N-(2-ethylhexyl)methylaminobenzoic acid ester of 2,4-dihydroxy-benzophenone, 4-N,N-(2-ethylhexyl)methylaminobenzoic acid ester with 4-hydroxydibenzoylmethane, 4-N,N-(2-ethylhexyl)methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, and mixtures thereof.

Generally, the sunscreens can comprise from about 0.5% to about 20% of the compositions useful herein. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

The compositions of the present invention can additionally comprise from about 0.1% to about 5% by weight of aluminium starch octenylsuccinate. Aluminium starch octenylsuccinate is the aluminium salt of the reaction product of octenylsuccinic anhydride with starch and is commercially available under the trade name from Dry Flo National Starch & Chemical Ltd. Dry Flo is useful herein from the viewpoint of skin feel and application characteristics. Other optional materials herein include pigments which, where water-insoluble, contribute to and are included in the total level of oil phase ingredients. Pigments suitable for use in the compositions of the present invention can be organic and/or inorganic. Also included within the term pigment are materials having a low color or lustre such as matte finishing agents, and also light scattering agents. Examples of suitable pigments are iron oxides, acyglutamate iron oxides, ultramarine blue, D&C dyes, carmine, and mixtures thereof. Depending upon the type of composition, a mixture of pigments will normally be used. The preferred pigments for use herein from the viewpoint of moisturisation, skin feel, skin appearance and emulsion compatibility are treated pigments. The pigments can be treated with compounds such as amino acids, silicones, lecithin and ester oils.

The pH of the compositions is preferably from about 4 to about 9, more preferably from about 6 to about 8.0.

The balance of the composition is water or an aqueous carrier suitable for topical application to the skin. The water content of the compositions herein is generally from about 30% to about 98.89%, preferably from about 50% to about 95% and especially from about 60% to about 90% by weight.

The invention is illustrated by the following examples

### EXAMPLES I to V

| | I/% | II/% | III/% | IV/% | V/% |
|---|---|---|---|---|---|
| Cetyl Alcohol | 0.72 | 0.5 | 0.8 | 0.65 | 0.75 |
| Stearic Acid | 0.11 | 0.2 | 0.1 | 0.2 | 0.1 |
| Steareth 100 | 0.1 | 0.1 | 0.15 | 0.15 | 0.15 |
| Propyl Paraben | 0.17 | 0.08 | 0.07 | 0.15 | 0.07 |
| Arlatone (RTM) | 1.0 | 2.0 | 1.5 | 1.0 | 4.0 |
| 2121(1) | | | | | |
| Glycerin | 3 | 4 | 8 | 2.5 | 3.5 |
| Carbopol (RTM) | 0.1 | 0.075 | 0.08 | 0.075 | 0.075 |
| 1382 | | | | | |
| Carbopol (RTM) 954 | 0.7 | 0.56 | 0.5 | 0.65 | 0.45 |
| Na4 EDTA | 0.1 | 0.2 | 0.1 | 0.1 | 0.1 |
| Methyl Paraben | 0.2 | 0.2 | 0.175 | 0.175 | 0.175 |
| NaOH (40% solution) | 1.0 | 0.8 | 0.8 | 0.8 | 0.8 |
| Dimethicone Q21403 | 1.0 | 1.0 | 0.5 | 2.0 | 1.0 |
| TiO₂ | 0.15 | 0.15 | 0.15 | 0.15 | 0.15 |
| Perfume | 0.2 | 0.2 | — | 0.2 | — |
| Urea | 2.5 | 1.5 | 3 | 2 | 2.5 |
| SEFA(2) | 0.0 | 0.0 | 2.5 | 2.0 | 2.2 |
| Octyldodecyl | 0.0 | 0.0 | 0.0 | 1.0 | 1.0 |
| benzoate | | | | | |
| KSG-18(3) | 3.0 | 1.5 | 2.0 | 3.2 | 2.5 |
| Colour | 0.0004 | 0.0002 | 0.0003 | 0.0 | 0.0 |
| Water | to 100 | to 100 | to 100 | to 100 | to 100 |

(1)Supplied by ICI
(2)Liquid sucrose polyester which is a mixture of hexa-, hepta-, and octa- sucrose esters esterified with mixed cottonseed oil fatty acids, predominately the octa-ester.
(3)Supplied by Shinetsu Chemical Co. Ltd.

The compositions are made as follows:

A first premix of thickening agents, Arlatone 2121, methyl paraben, glycerine/TiO2 premix, and other water soluble ingredients apart from urea, is prepared by admixing in water and heating to about 80° C. A second premix of oil phase ingredients other than silicone gum is prepared by mixing and heating and is added to the aqueous premix.

The resulting mixture is cooled to about 60° C. The silicone gum, KSG-18 and urea are then added to the resulting oil-in-water emulsion and the mixture is cooled before adding minor ingredients. The composition is ready for packaging.

The compositions display improved moisturisation, skin feel and skin care characteristics together with reduced greasiness and excellent rub-in and fast absorption characteristics.

1. A skin care composition comprising:
(a) a silicone-containing phase comprising crosslinked polyorganosiloxane polymer and silicone oil, wherein the composition comprises from about 0.1% to about 20% by weight of the combination of crosslinked polyorganosiloxane polymer and silicone oil;
(b) from about 0.1% to about 20% by weight of an organic liquid crystal-forming amphiphilic surfactant; and
(c) water;
wherein the composition is in the form of an oil-in-water emulsion.

2. A composition according to claim 1 comprising from about 0.5% to about 10%, preferably from about 0.5% to about 5%, by weight of composition, of the combination of crosslinked polyorganosiloxane polymer and silicone oil.

3. A composition according to claim 1 or 2 wherein the combination of crosslinked polyorganosiloxane polymer and silicone oil consists of from about 10% to about 40%, preferably from about 20% to about 30%, by weight of the combination, of the crosslinked polymer and from about 60% to about 90%, preferably from about 70% to about 80%, by weight of the combination, of the silicone oil.

4. A composition according to any of claims 1 to 3 wherein the composition comprises crosslinked polyorganosiloxane polymer comprises polyorganosiloxane polymer crosslinked by a crosslinking agent, wherein the crosslinking agent has the formula: 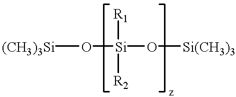
wherein R₁ is methyl, ethyl, propyl or phenyl, R₂ is H or —(CH₂)ₙCH═CH₂ and z is in the range of from about 1 to about 1000.

5. A composition according to claim 4 wherein the crosslinking agent has the formula: 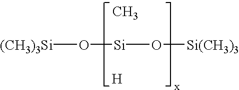
wherein x is in the range of from about 1 to about 1000.

6. A composition according to claim 4 or 5 wherein the crosslinked polysiloxane polymer comprises from about 10% to about 50%, preferably from about 20% to about 30%, by weight the crosslinked polysiloxane polymer, of crosslinking agent.

7. A composition according to any of claims 4 to 6 wherein the polyorganosiloxane polymer is selected from polymers having the general formula: 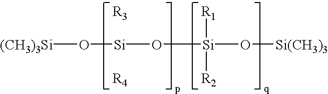
wherein R₁ is methyl, ethyl, propyl or phenyl, R₂ is H or —(CH₂)ₙCH═CH₂, R₃ and R₄ are independently selected from methyl, ethyl, propyl and phenyl, p is an integer in the range of from about 1 to about 2000, q is an integer in the range of from about 1 to about 1000.

8. A composition according to claim 7 wherein the polyorganosiloxane polymer is selected from polymers having the formula: 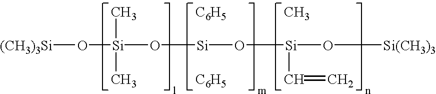
wherein l is an integer in the range of from about 1 to about 1000, m is an integer in the range from 0 to about 1000 and n is an integer in the range of from about 1 to about 1000.

9. A composition according to claim 7 wherein m is in the range of from about 1 to about 1000, preferably from about 200 to about 800.

10. A composition according to any of claims 1 to 9 wherein the silicone oil is selected from silicone oils having a weight average moleular weight of about 100,000 or less, preferably about 50,000 or less, more preferably selected from silicone oils having a weight average molecular weight in the range from about 100 to about 50,000, especially from about 200 to about 40,000.

11. A composition according to any of claims 1 to 10 wherein the silicone oil is selected from dimethicone, decamethylcyclopentasiloxane, octamethylcyclotetrasiloxane and phenyl methicone, and mixtures thereof.

12. A composition according to any of claim 1 to 10 wherein the silicone oil is phenyl methicone.

13. A skin care composition according to any of claims 1 or 12 wherein the amphiphilic emulsifier is selected from polyol esters, alkoxylated polyol esters and mixtures thereof, said esters preferably being selected from mono-, di- and triester materials.

14. A skin care composition according to any of claims 1 to 13 wherein the organic amphiphilic emulsifier is a blend of sorbitan stearate and sucrose cocoate.

15. A skin care composition according to any of claims 1 to 14 comprising from about 0.01% to about 20% by weight of a liquid, polyol carboxylic acid ester having a polyol moiety and at least 4 carboxylic acid moieties, wherein the polyol moiety is selected from sugars and sugar alcohols containing from about 4 to about 8 hydroxyl groups, and wherein each carboxylic acid moiety has from about 8 to about 22 carbon atoms, and wherein said liquid polyol carboxylic acid ester has a complete melting point of less than about 30° C.

16. A composition according to claim 15 wherein said liquid polyol carboxylic acid ester contains no more than about 2 free hydroxyl groups.

17. A composition according to claim 15 or 16 wherein said carboxylic acid moieties contain from about 14 to about 18 carbon atoms.

18. A composition according to any of claims 15 to 17 wherein said polyol moiety is selected from erythritol, xylitol, sorbitol, glucose, sucrose, and mixtures thereof.

19. A composition according to any of claims 15 to 18 wherein said polyol moiety is sucrose.

20. A composition according to any of claims 15 to 19 wherein said liquid polyol carboxylic acid ester has a complete melting point below about 27.5° C.

21. A composition according to any of claims 15 to 20 wherein said liquid polyol carboxylic acid polyester has a complete melting point below about 25° C.

22. A composition according to any of claims 15 to 21 wherein said liquid carboxylic acid polyol ester is selected from sucrose pentaoleate, sucrose hexaoleate, sucrose heptaoleate, sucrose octaoleate, and mixtures thereof.

23. A skin care composition according to any of claims 1 to 22 additionally comprising from about 0.1% to about 20% by weight of urea.

24. A skin care composition according to any of claims 1 to 23 additionally comprising from about 0.1% to about 20% by weight of a humectant selected from glycerine, polyglycerylmethacrylate lubricants, butylene glycol, sorbitol, panthenols, propylene glycol, hexylene glycol, ethoxylated glucose derivatives, hexanetriol and glucose ethers, and mixtures thereof.

25. A skin care composition according to claim 24 wherein the humectant is glycerine.

26. A skin care composition comprising:
(a) a first silicone-containing phase comprising crosslinked polyorganosiloxane polymer and silicone oil, wherein the composition comprises from about 0.1% to about 20% by weight of the combination of crosslinked polyorganosiloxane polymer and silicone oil;
(b) a second non-crosslinked silicone-containing phase and
(c) a third oil phase comprising natural or synthetic oil selected from mineral, vegetable, and animal oils, fats and waxes, fatty acid esters, fatty alcohols, fatty acids and mixtures thereof; and
(d) water;
wherein the composition is in the form of an oil-in-water emulsion.
